(19) United States
(12) Patent Application Publication
(10) Pub. No.: US 2026/0165633 A1
(43) Pub. Date: Jun. 18, 2026

(54) SYSTEM AND METHOD FOR TRAINING A SUBJECT TO SELF-REGULATE NEURAL VARIABILITY

(71) Applicant: CARNEGIE MELLON UNIVERSITY, Pittsburgh, PA (US)

(72) Inventors: RYAN WILLIAMSON, Pittsburgh, PA (US); AKASH UMAKANTHA, Pittsburgh, PA (US); CHRIS KI, Pittsburgh, PA (US); MATTHEW SMITH, Pittsburgh, PA (US); BYRON YU, Pittsburgh, PA (US)

(73) Assignee: CARNEGIE MELLON UNIVERSITY, Pittsburgh, PA (US)

(21) Appl. No.: 19/122,972

(22) PCT Filed: Jan. 9, 2024
(86) PCT No.: PCT/US2024/010801

§ 371 Date: Apr. 21, 2025

Related U.S. Application Data
(60) Provisional application No. 63/439,314, filed on Jan. 17, 2023.

(51) Int. Cl. A61B 5/375; A61B 5/00; A61B 5/37; A61B 5/378

(52) U.S. Cl. CPC A61B 5/375; A61B 5/37; A61B 5/378; A61B 5/7264; A61B 2503/40; A61B 2505/09; A61B 2560/0238

(57) ABSTRACT

Disclosed herein is a system and method for training a subject to self-regulate neural variability. The system and method implements a prefrontal cortex brain-computer interface (BCI) and a method that allows subjects to use neurofeedback to produce a desired neural activity by regulation of their arousal levels to stabilize their neural activity across a timescale of seconds or minutes. The system comprises a prefrontal cortex brain-computer interface (BCI) to train subjects to use neurofeedback to produce desired neural activity. Subjects used the disclosed system and method, which includes BCI feedback, to self-regulate their arousal levels and successfully stabilize their neural activity.

### RELATED APPLICATIONS

This application is a national filing under 35 U.S.C. § 371 claiming the benefit and priority to International Patent Application No. PCT/US24/10801, filed Jan. 9, 2024 entitled “SYSTEM AND METHOD FOR TRAINING A SUBJECT TO SELF-REGULATE NEURAL VARIABILITY”, which claims the benefit of U.S. Provisional Patent Application No. 63/439,314 filed Jan. 17, 2023, the contents of which are incorporated herein in their entireties.

### GOVERNMENT INTEREST

This invention was made with the support of the United States government under contracts 1734916 and 1954107 awarded by the National Science Foundation (NSF). The U.S. government has certain rights in the invention.

### BACKGROUND OF THE INVENTION

All activities are associated with a neural pattern of activity. For example, consider shooting free throws on a basketball court. Even if the shooter tries the same shot each time, their internal states, such as arousal and motivation, may change from shot to shot. Such changes in cognitive states over time may manifest as neural variability, in which the brain produces variable activity when presented with the same task conditions multiple times. These internal state fluctuations are captured in neural population activity and indirectly shape performance. For instance, slow fluctuations in the prefrontal cortex areas corresponded to an arousal-related signal that reflected the impulsivity of the subject during a change detection task.

Deficits in regulating neural variability have been linked to neuropsychiatric disorders. Understanding the degree to which one could control neural variability over time has implications for enhancing and restoring the brain's cognitive capabilities. Therefore, it would be desirable to be able to train a subject to regulate this neural variability.

### SUMMARY OF THE INVENTION

When presented with identical task conditions across trials, the brain produces variable patterns of neural activity, wherein the variability stems from many sources, one of which is fluctuations in internal states (e.g., arousal). Reducing neural variability has implications for improving the brain's cognitive abilities.

Disclosed herein is a system and method implementing a prefrontal cortex brain-computer interface (BCI) that allows subjects to use neurofeedback to produce a desired neural activity by regulation of their arousal levels to stabilize their neural activity across a timescale of seconds or minutes. The system comprises a prefrontal cortex brain-computer interface (BCI) that trains subjects to use neurofeedback to produce desired neural activity. Subjects used the disclosed system and method, which includes BCI feedback, to self-regulate their arousal levels and successfully stabilize their neural activity.

Through moment-to-moment neurofeedback, the BCI framework offers subjects precise temporal control of their neural activity. The disclosed brain-computer interface (BCI) task utilizes a metric, termed the neural distance, to characterize how far the current neural population activity is from a baseline state. Using the disclosed system and method, subjects can be trained to reduce their neural distance. Subjects reduce their neural distances by relying on accurate moment-to-moment neurofeedback to self-regulate an internal state, possibly linked to arousal.

### BRIEF DESCRIPTION OF THE DRAWINGS

FIG. 1 is an illustration of the variability of brain activity even when task variables are kept constant,

FIG. 2 is an illustration of the use of neurofeedback from a brain-computer interface to subdue variability over time.

FIG. 3 is a block diagram illustrating the calibration task and the brain-computer interface task.

FIG. 4 is a block diagram illustrating brain-computer interface trials, which show genuine neurofeedback, and sham trials, which show realistic but inaccurate visual feedback.

FIG. 5 is an illustration of determining if sham trials are correct or incorrect post hoc.

FIG. 6 is a scatterplot of the bin-averaged neural distances across trials of an example session.

FIG. 7 is a graph showing a comparison of correct percentages for BCI block and sham block trials.

FIG. 8 is a graph showing a comparison of trial-averaged neural distances for BCI block and sham block trials.

FIG. 9 is a scatterplot illustrating an example session (same as the session shown in in FIG. 6) showing each trial's bin-averaged neural distance across trials in a session.

FIG. 10 is a graph showing a comparison of linear regression slopes for sham and BCI (matched in clock time) blocks.

FIG. 11 is an Illustration showing the differences between interleaved sham and block sham trials. This comparison is important for demonstrating that animals used moment-to-moment visual feedback to control its neural activity.

FIG. 12 is a graph showing a comparison of % correct trials for BCI and interleaved sham trials.

FIG. 13 is a graph showing a comparison of average neural distances for BCI and Interleaved sham trials.

FIG. 14 graphically shows one possible mechanism by which the subject may suppress neural variability over time through the control of an internal state.

FIG. 15 is a graph showing significantly positive correlations between pupil sizes and shown annulus sizes during BCI trials.

FIG. 16 graphically shows that the correlations observed in BCI trials cannot be explained by luminance-driven changes brought on by the size-varying annulus.

FIG. 17 is a graph showing that during sham trials, predicted annulus sizes have more positive session-wide correlations with pupil sizes than shown annulus sizes.

FIG. 18 is a flow chart depicting the calibration task.

FIG. 19 is a flow chart depicting the BCI task.

### DETAILED DESCRIPTION

Disclosed herein is a system and method for training a subject to moderate their neural responses to stimuli to minimize neural variability over time. The system and method were implemented using rhesus macaque monkeys as subjects but as would be realized, the system and method can be used with any subject, including humans.

In one embodiment, two adult male rhesus macaque monkeys were surgically implanted with titanium headposts, which were fixed onto the skull of each subject to limit head movement and facilitate eye movement tracking and recording of neural activity. Subjects were then implanted with an electrode array. In one embodiment, the electrode array is a 96-electrode “Utah” array manufactured by Blackrock Microsystems of Salt Lake City, UT, although other electrode arrays could be used. The electrode array was placed in the prefrontal cortex anterior to the arcuate sulcus and dorsal to the medial sulcus in both hemispheres. Other positionings of the array are contemplated to be within the scope of the invention.

The subjects were head-fixed and positioned 36 cm from a 21-in cathode ray tube monitor with 1024×768 pixel resolution and a refresh rate of 100 Hz. Task feedback was displayed using custom software that utilized psychophysics toolbox extensions. In one embodiment, the custom software may be based on in Matlab from MathWorks of Natick, MA. Eye position was monitored using monocular infrared tracking at a 1000 Hz sample rate. In one embodiment, eye tracking as performed using EyeLink 1000 from RS Research of Mississauga, Ontario, Canada).

Neural activity was recorded from an implanted microelectrode array in area 8ar of the dorsolateral prefrontal cortex (dLPFC). Signals from the implanted electrodes were band-pass filtered (0.3-7500 Hz) and then digitized at 30,000 Hz before being stored offline for analysis. For each electrode, spiking waveforms were defined as a 52-sample (1.73 ms) window of the filtered voltage signal triggered by the signal crossing a predefined threshold. The threshold was defined as a multiple of the root-mean-square voltage of a brief epoch of the raw signal on each electrode collected at the beginning of the session. All behavioral and neural data was recorded for further offline processing and analysis. In one embodiment, the recording was performed using a Grapevine recording system from Ripple of Salt Lake City, UT.

To separate waveforms likely to be caused by neural spiking from waveforms caused by other electrical artifacts, a neural network classifier trained to classify spike waveforms as “neural” or “not neural” was used. The classifier was trained using array recordings from multiple subjects in which the waveforms had been hand sorted. In one embodiment, the classifier successfully removes a high percentage of putative noise waveforms in held-out test waveform datasets. Classification required very little computation time, allowing for the classification of hundreds of waveforms in a few milliseconds. This algorithm was applied to the prefrontal cortex recordings online to ensure that activity going into the BCI was of neural origin.

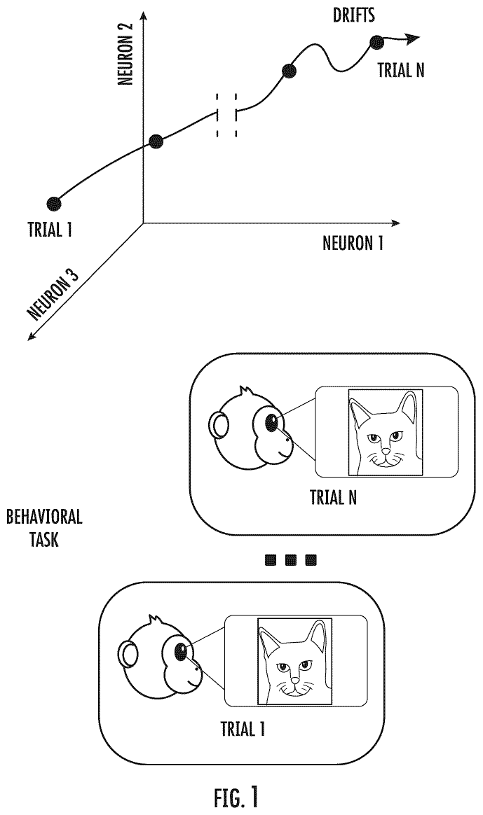

Because neurons in the prefrontal cortex encode direction-specific sensory representations of visual stimuli, in one embodiment, visual feedback was provided to the subject. In one embodiment, to prevent the BCI from being spatially biased, the visual feedback during trials with neurofeedback comprised the display of an annulus wherein the diameter of the annulus could be controlled by the neural activity of the subject. To represent how far a subject's current neural activity was from a target neural state (FIG. 1), the BCI offered one-dimensional feedback, as neural activity directly controlled the diameter of the annulus.

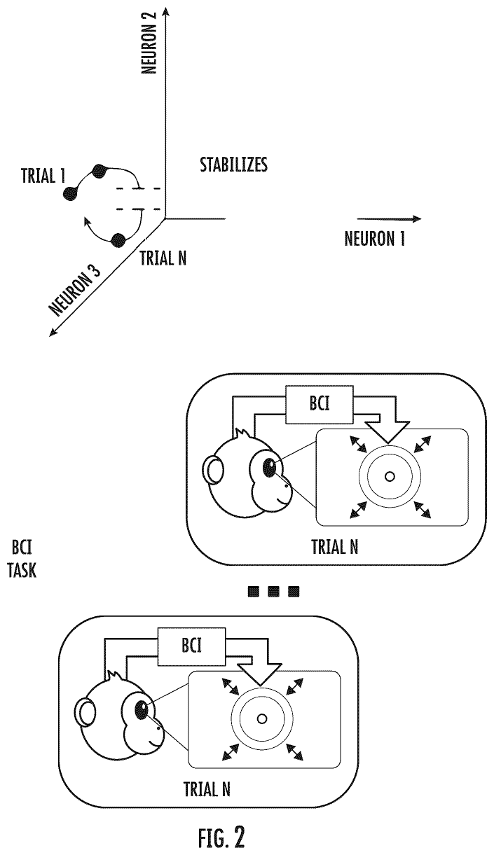

FIG. 1 shows that, even when task variables are kept constant over time in a behavioral task, the brain produces different activity patterns. The graph shows how neural population activity can differ across trials even when the subject is shown the same sensory stimuli (e.g., the cat in trials 1 . . . N). These changes can be induced by variations in the subject's internal state (e.g., arousal). FIG. 2 shows the BCI feedback paradigm in which we provide visual neurofeedback is provided, and the graph shows wherein the neural variability can be controlled as the neural activity is used to drive the annulus to a target state.

In one embodiment the target state is the reduction of the diameter of the annulus toward the central dot. As would be realized by one of skill in the art, the stimulus is not limited a visual stimulus, nor to the specific visual stimulus described herein, but may be any sensory stimulus, for example, other visual stimuli, audible stimuli, haptic stimuli, etc.

In the exemplary embodiment described herein, the visual feedback was constrained to lie outside the receptive fields of the recorded neurons by having the annulus size range from 10 pixels to 80 pixels. In all experimental sessions, the subjects performed a calibration task and a brain-computer interface (BCI) task.

The calibration task comprised exposing the subjects to a set of 60 calibration trials. In a calibration trial, a circle was displayed at the center of the screen against an isoluminant background. A trial was initiated when the subject fixated on the blue circle, at which point an annulus appeared on the screen centered on the fixation circle. The annulus remained fixed in size until 400 ms post-fixation, after which the annulus started shrinking. The diameter of the annulus during these trials was limited to lie between 10 and 80 pixels. Over the next 3.4 s, the subject simply had to maintain fixation while the radius of the annulus shrank at a fixed speed. During this entire time, the annulus remained centered on the fixation dot. The subject was rewarded if it successfully maintained fixation throughout the entire 3.4 s period during which the annulus shrank.

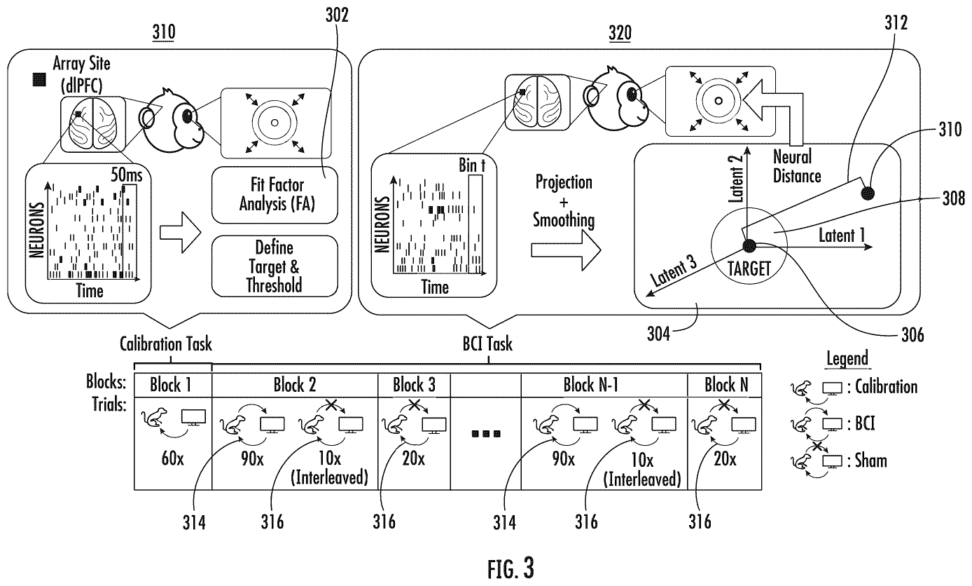

The calibration task is depicted in FIG. 3. Neural activity was recorded using the implanted multi-electrode array. During the calibration task, the subjects fixated on a central dot on an isoluminant screen while an annulus shrunk toward the center. A factor analysis model 302 was then fit onto binned spike counts from the calibration trials to identify a low-dimensional subspace 304, a target state 306 and a reward threshold 308. During the BCI task, subjects were shown the visual neurofeedback in the form of an annulus that was updated every 50 ms. The neural distance, which is the Euclidean distance 312 of the current neural state 310 to the target state 306 in low-dimensional subspace 304, was used to determine the diameter of the annulus to be shown in the next time step. In the BCI task, subjects completed alternating blocks of BCI trials 314 and sham trials 316. BCI blocks each contained 90 BCI trials 314 and 10 randomly interleaved sham trials 316 while sham blocks each contained 20 consecutive sham trials 316.

The calibration task 1800 is depicted in flowchart form FIG. 18. At step 1802, the subject is exposed to the stimulus. At step 1804 the stimulus is maintained in a fixed state for a predetermined period of time. For example, when the stimulus is the visual display of the annulus, the annulus is kept at a constant diameter during the fixation period. At step 1806 the stimulus is altered during the series of time steps and, at step 1808, the neural activity of the subject is recorded as the stimulus changes. The process is repeated a predetermined number of times.

Taking the recorded prefrontal cortex (PFC) neural activity from the calibration trials, a light sorting was first performed using a neural network sorter. The spike counts were binned into non-overlapping 50 ms bins beginning 400 ms after fixation to the end of the 3.5 s period in which the annulus moved on its own. The binned spike counts were then concatenated across all 60 calibration trials and factor analysis (FA) was applied to the aggregate spike count matrix to identify a low-dimensional BCI subspace that best described shared variance at the population level. Factor analysis is defined by the following model:

$$x ∼ ( d , {LL}^{T} + Ψ ) \qquad ( 1 )$$

where:
x∈ [symbol] ^{p} is a vector of spike counts across the p simultaneously-recorded neurons;
d∈ [symbol] ^{p} is a vector of mean spike counts;
L∈ [symbol] ^{p×m} is the loading matrix relating m latent variables to the neural activity; and
Ψ∈ [symbol] ^{p×p} is a diagonal matrix of independent variances for each neuron.

For the BCI task, the number of latent variables was always set to either 4 or 5 depending on the subject (Subject S: 5, Subject P: 4). The model parameters μ, L and Ψ were estimated using the expectation-maximization (EM) algorithm.

For a binned spike count vector at time step t from calibration trial i (denoted as x_{i,t}∈ [symbol] ^{p}), its projection into the m-dimensional BCI subspace was found by computing the posterior mean [symbol] ∈ [symbol] ^{m}:

$$= [ {z}_{i , t} \vert {x}_{i , t} ] = {{L}^{T} ( {LL}^{T} + Ψ )}^{- 1} ( {x}_{i , t} - d ) \qquad ( 2 )$$

For each calibration trial i, exponential smoothing was applied across time bins with a smoothing factor of α∈[0,1] to get the smoothed latent w_{i,t}∈ [symbol] ^{m}:

$${w}_{i , t} = \{ \begin{matrix} & if t - 0 \\ α + ( 1 - α ) {w}_{i , t - 1} & else\end{matrix} \qquad ( 3 )$$

For a bin at time step t (other than the 1st), the smoothed value w_{i,t} was a weighted average of all observations from calibration trial i up to time step t with the weights exponentially decaying over time. α thus determines the weight of the current latent z_{i,t} for the weighted average w_{i,t} (i.e., a low α corresponds to heavy smoothing/reliance on past neural activity). In all experiments, α=0.1535 was used.

The exponential smoother was reset at the beginning of each calibration trial such that the smoothed latent for the first bin of each trial was simply its posterior mean. The target baseline state μ is defined as the mean of the smoothed FA projections:

$$μ = \frac{1}{nT} {\sum }_{i = 1}^{n} {\sum }_{t = 1}^{T} {w}_{i , t} \qquad ( 4 )$$

where:
n is the number of calibration trials performed; and
T=68 (matches the 3.4 s period in which the annulus shrunk on its own) refers to the number of 50 ms time bins in each calibration trial.

In all sessions, μ, was very close to the origin. This is due to a property of the FA model in which the mean of the projections for the training data (all bins in the calibration trials) is the zero vector:

$$β = \frac{1}{nT} {\sum }_{i = 1}^{n} {\sum }_{t = 1}^{T} = 0 \qquad ( 5 )$$

This slight deviation of μ from 0 stems from the light smoothing that was applied to the projections before computing the mean.

The neural distance for bin t of calibration trial i, y_{i,t}, which is the squared Euclidean distance of a smoothed FA latent at time bin t from the target state, was then computed for all time bins in the calibration trials:

$${y}_{i , t} = {‖ {w}_{i , t} - μ ‖}^{2} \qquad ( 6 )$$

The neural distance is thus a measure of how far the current smoothed neural state, w_{i,t}, is from the mean smoothed activity μ observed at the beginning of a session. To determine the distance reward threshold 308 that would achieve a reward in a BCI trial, all distances were aggregated, and percentiles computed in 0.1 percentile increments. Percentile values were then swept to determine the percentile of the distance that would lead the subject to get 50% of the calibration trials being correct if treated as BCI trials. This distance percentile was then set as the reward threshold for the rest of the session. The value of 50% was selected such that the subject would not find it too difficult to perform the BCI task.

To provide further intuition into what neural distance measures, neural distance is decomposed into a sum involving a weighted average of the deviation of each neuron's current firing rate from its mean firing rate during the calibration trials. Within a single trial, this weighted average term approximated the neural distance very well over time. In a crude sense, one can view reducing the neural distance as trying to get each recorded neuron's firing rate as close as possible to the same neuron's mean firing rate observed at the start of an experiment.

After the calibration task, the subject performed a BCI task. In the BCI task, the subject was presented with alternating BCI trials and sham blocks of trials 316. BCI blocks mostly contained BCI trials and had rare but randomly interleaved sham trials (Subject P: 90 BCI, 10 interleaved sham trials; Subject S: 80 BCI, 20 interleaved sham trials). Sham blocks always contained 20 sham trials, as shown in FIG. 3.

Like calibration trials, BCI and sham trials both began with a 400 ms fixation period during which the annulus remained still. Then, the feedback period followed, and based on the trial type, different feedback was shown. In BCI trials, the subject was given visual neurofeedback such that the annulus size correctly reflected how far the subject's current neural activity was from the target state. During these trials, the subject's objective was to have its neural activity be within the reward threshold (this corresponds to keeping the annulus diameter below 30 pixels) for at least 400 ms. If the subject achieved an annulus size below the reward threshold for 400 ms (i.e., 8 consecutive bins), the trial would end with the subject receiving a liquid reward. If the subject never reached the success condition during the 3.4 s feedback period, the trial ended with the subject not receiving a reward.

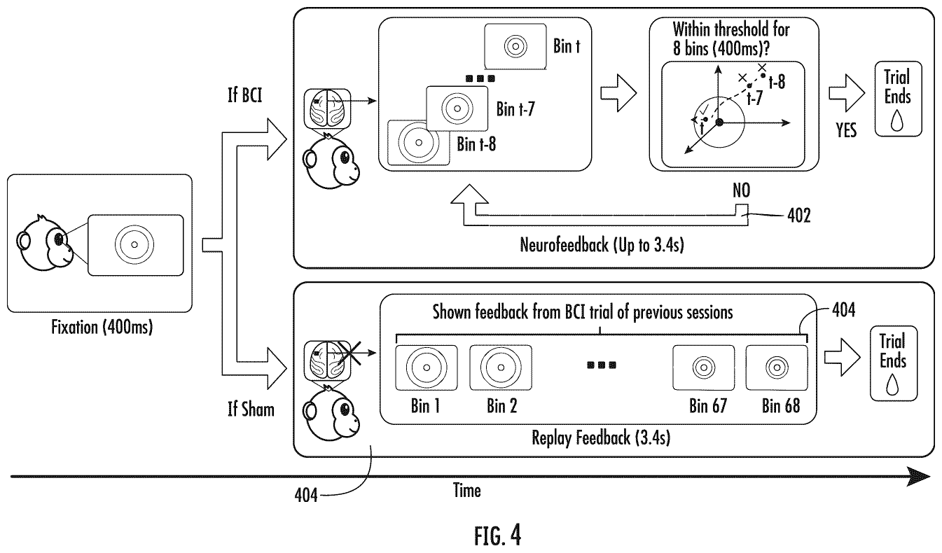

The BCI task is shown graphically in FIG. 4. In both BCI and sham trials, the subjects first fixated a central blue dot for 400 ms. Then, in BCI trials, for the next 3.4 s, accurate visual neurofeedback 402 was provided to the subject. If the subject kept its neural distance within the reward threshold for 400 ms, the trial was considered correct and ended. If not, the trial continued until 3.4 s passed. Sham trials always lasted 3.4 s and showed visual feedback displayed during correct BCI trials sampled from previous sessions 404. As a result, independent of their neural activity, the subjects always received rewards.

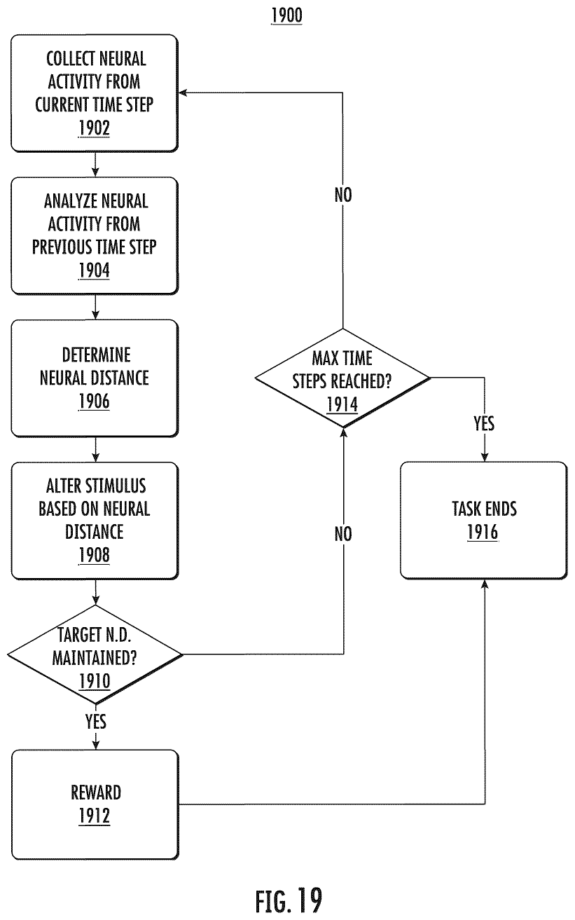

The BCI task is depicted in the flowchart form in FIG. 19. At step 1902, the neural activity from the current time step is collected and recorded. At step 1904, the neural activity from the previous time step is analyzed and, at step 1906, the neural distance, as described herein, is determined. At step 1908, the stimulus is altered based on the calculated neural distance. For example, using the visual stimulus described herein, for a larger neural distance, the diameter of the annulus is enlarged, and, for a smaller neural distance, the diameter of the annulus is reduced. Step 1910, it is determined if the neural distance has been maintained within the target threshold for a predetermined number of time steps. That is, the diameter of the annulus is within a target range for a predetermined period of time. If so, a reward is issued in step 1912 and the trial ends at step 1960. If not, it is determined if the maximum number of time steps has been reached at step 1914 and, if not, the process repeats. If the maximum number of time steps been reached at step 1914, the task ends at step 1960 without the subject being rewarded.

In sham trials, the feedback was inconsistent with the subject's current neural state. Unlike BCI trials, in sham trials, the feedback period lasted the full 3.4 s and as long as subjects maintained fixation throughout that time, they were rewarded. Only completed BCI and sham blocks were kept for analysis.

In a BCI trial, the size of the annulus at time bin t, aₜ, was directly determined by the neural activity observed during the previous bin t−1. Similar to how the neural distance was found for the BCI calibration phase, the spikes from the previous 50 ms were sorted using the neural network sorter, the resulting spike count vector was projected into the calibration-defined factor space and the neural distance was computed in accordance with Eq. (6). The neural distance was mapped to a percentile value using the percentiles defined during the calibration task. For all BCI trials in a session, the μ defined during the session's calibration trials was used to compute the neural distance.

This percentile value was then mapped to an annulus size using a predefined affine transformation.

Although the annulus only changed sizes during the feedback period, smoothed neural distances were computed and tracked for bins during the fixation period (first 8 50 ms bins of a trial). This “freeze” period provided enough time for the trial's exponential smoother to stabilize. Thus, when the neurofeedback period started at the 9th bin of a trial, the smoothed distance calculated for bin 8, w₈, was used to determine the annulus that was displayed. The annulus was updated every 50 ms to reflect the annulus size computed from the previous 50 ms.

The description that follows applies to both interleaved and block sham trials. After the initial 400 ms fixation period, subjects viewed replay feedback from a test session's BCI trial that lasted the full 3.4 s of the feedback period. As long as the subjects maintained fixation during the replay feedback, they were given a liquid reward. To ensure the subject did not break the association between shrinking the annulus and receiving a reward, correct BCI trials were specifically selected in which the subject met the success criteria right at the end of the 3.4 s feedback period. At the beginning of each day, a set of 5 BCI trials that met this requirement from a test session were determined. Then, during a sham trial, the visual feedback associated with a BCI trial from this set was randomly selected and shown. The feedback during the sham trials was unaffected by the current state of the subject's neural activity.

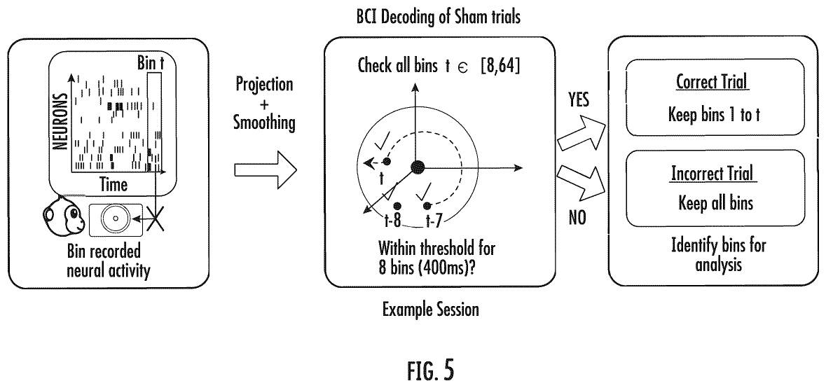

To compare a session's BCI and sham trials on BCI task performance, recorded neural activity during sham trials was passed through that session's BCI decoder in an offline setting. For each sham trial (interleaved and block sham), the recorded neural activity was binned into 50 ms time bins, the spike counts were projected into FA space in accordance with Eq. (2), exponential smoothing was applied in accordance with Eq. (3), and the neural distances were computed in accordance with Eq. (6). If, at any point during the feedback period (8th to 64th time bin in a trial), the success conditions were met (FIG. 5) the trial was truncated such that only neural activity from bin 1 to bin t, the earliest bin at which this success criterion was met, was considered for offline analysis. This processing is equivalent to what neural activity during BCI trials underwent in an experimental session. For sham trials, it was found that the computed average neural distances increased when we included all the time bins. This truncation scheme of sham trials thus provided a stronger null for assessing the extent to which subjects could use the BCI.

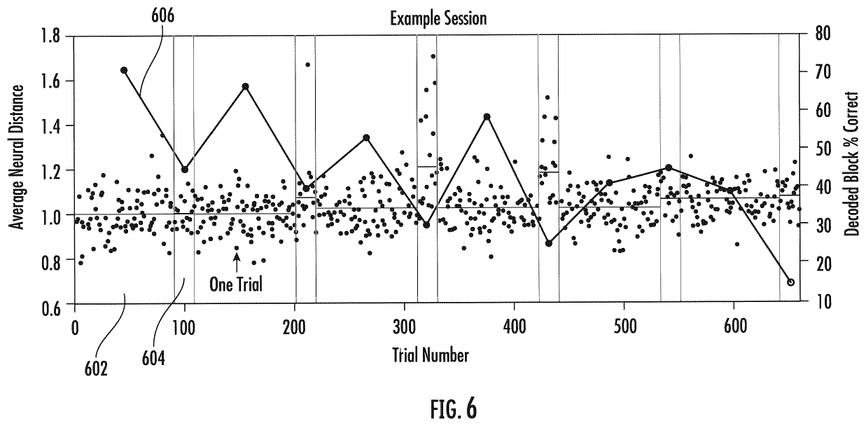

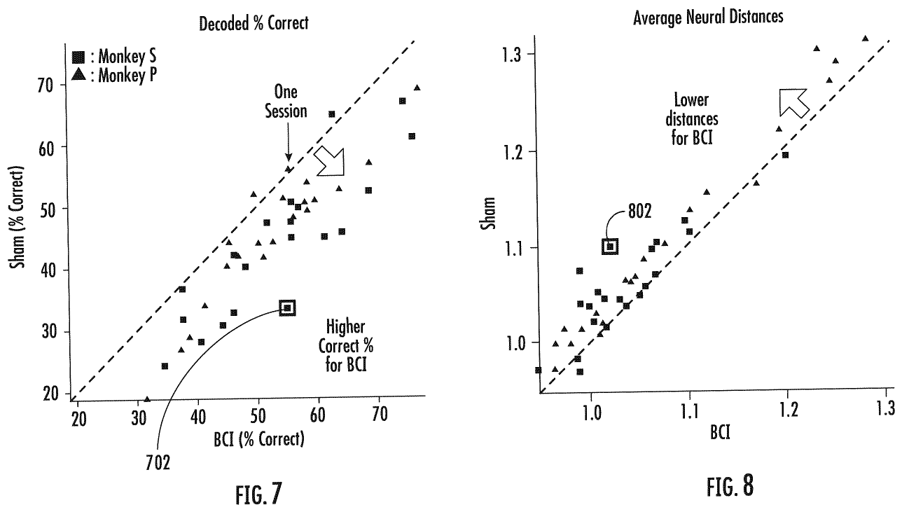

As shown in FIG. 5, sham trials are determined to be correct or incorrect post hoc by passing neural activity recorded during the sham trials through the session's BCI decoder. Sham trials were only truncated if they were considered correct by the BCI decoder. All subsequent analyses make comparisons between BCI trials and sham trials using the bins identified through this preprocessing step. FIG. 6 is a scatterplot of each trial's bin-averaged neural distances (computed offline) from an example session from subject S. The differently shaded regions denote the two block conditions: BCI (602) and sham (604). The correct percentage 606 for each block is shown. The horizontal lines indicate the average bin-averaged neural distance observed for that block. Interleaved sham trials are not plotted nor are they included for the block % calculation of BCI blocks. This session had the largest difference in decoded correct percentage between BCI block (55%) and sham block (33%) trials. FIG. 7 is a graph comparing correct percentages (computed as the number of correct trials in session divided by the number of total trials for condition in session) for BCI block and sham block trials. BCI blocks have higher correct percentages than sham blocks. The box 702 indicates the session corresponding to the example session shown in FIG. 6. FIG. 8 is a graph of comparing trial-averaged neural distances for BCI block and sham block trials. The average neural distance for each condition was computed across all times bins of all trials in a session. BCI blocks had significantly lower average neural distances than sham blocks. Box 802 indicates the session corresponding to the example session shown in FIG. 6.

All neural analyses discussed for sham (interleaved and Block) and BCI trials are based on the kept bins of neural activity after the recorded neural activity has been passed through our decoder offline. For BCI trials, there is no difference between the remaining neural activity and recorded neural activity as during the experiment, BCI trials ended earlier if the success condition was met. To determine whether a sham trial was correct or not and what bins to consider for offline analysis, neural activity recorded during the sham trials was passed through the BCI decoder offline to match what occurred during the experiment for BCI trials.

During the experiments, smoothing was used in the neural distance computations to provide informative visual feedback and accommodate the online task demands of holding one's neural activity near the target for an extended period. However, for all the neural distances calculated from, temporal smoothing was excluded from the neural distance computations to focus on how the subject's neural activity changed on a moment-by-moment basis.

Specifically, the following equation was used to determine the neural distance at time bin t for a trial k:

$${y}_{k , t} = {‖ - β ‖}^{2} = {‖ ‖}^{2} \qquad ( 7 )$$

The target state is defined to be the mean neural activity observed during the calibration task. In this setting where smoothing is not included to find the neural distances, we thus want our target state to be the mean of the calibration trials' unsmoothed FA projections, which is determined by Eq. (5).

Because BCI blocks had more trials than sham blocks, every BCI block was truncated such that they were similar in clock time duration to their subsequent sham block (See FIG. 6). This matching of BCI blocks was performed using clock time duration rather than trial numbers because subjects sometimes took long pauses in between trials. To account for sham blocks with long breaks in between trials, the distribution of sham block clock time durations was computed and sham blocks with abnormally long durations were removed, along with their paired BCI blocks for this analysis.

When matching BCI blocks, the BCI blocks were end-aligned to their paired sham blocks and the last set of trials were selected such that the clock time duration of this set of trials matched the duration of the corresponding sham block. For instance, if a sham block lasted ˜90 s, the last n trials of paired BCI block were selected such that the n trials' total time duration was roughly 90 s. Trials were selected from the end of the BCI block as they were the closest in time to the sham block trials that they would be compared to. When fitting the linear regression models, the y-intercept was included in the model such that the model was described by the following relationship:

$$y = {β}_{0} + {β}_{1} x \qquad ( 8 )$$

wherein:
γ, β₀, β₁, x∈ [symbol] .

y represents the bin-averaged distance for a trial that occurred at clock time x. The β_{1,}s found after fitting the model to each block are computed. Then, the block-averaged β1's computed of the two conditions are compared for all sessions.

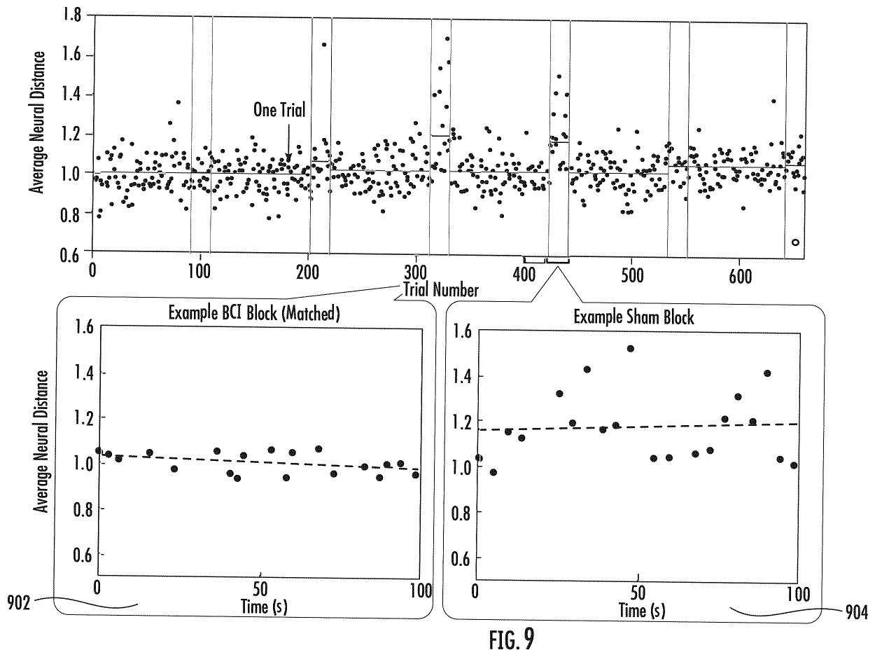

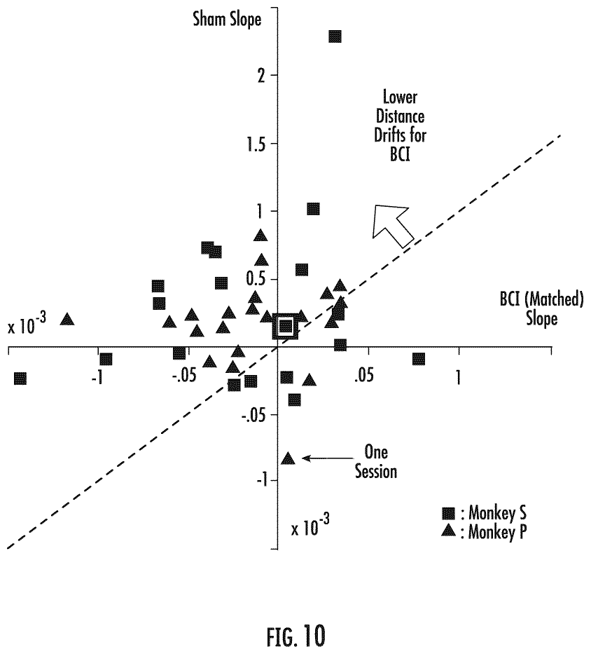

FIG. 9 shows an example session (same as session in FIG. 5) showing each trial's bin-averaged neural distance. An example BCI block 902 and sham block 904 have been selected from the example session shown directly above. BCI blocks have been matched in clock time duration (in seconds) to the adjacent sham block that follows them. The dashed lines represent linear regression lines that have been fit to bin-averaged neural distances over time. FIG. 10 is a graph showing a comparison of linear regression slopes for sham and BCI (matched) blocks. Each point represents a session-wide average computed across blocks after a regression slope is found for each block. The slopes for BCI blocks are significantly lower than the slopes for sham blocks.

Using the disclosed system and method, two male adult rhesus macaques were trained to control the size of an annulus on a screen by modulating the activity of ˜30 neural units in the PFC. Every 50 ms, the annulus size was updated to reflect how far the subject's current neural activity was from a baseline state observed at the start of the experiment. Subjects can successfully use the disclosed system and method to suppress fluctuations in their neural activity over time.

At the beginning of each experimental session, subjects performed a set of calibration trials in which they fixated a central dot while an annulus gradually shrunk towards the center. The neural activity recorded during these trials was used to define a BCI mapping that related neural activity to visual feedback for the subsequent BCI task. To do this, a factor analysis (FA) model (302 in FIG. 3) was fit to the recorded spike counts to identify a set of dimensions, or factors that captured dominant patterns of co-modulation across the neural population. BCI control of the annulus was only affected by changes in neural activity along these factors that best described shared variability in the neural population. In this low-dimensional subspace, we defined the target baseline state 306 as the average neural activity observed during the calibration task. During the BCI task 320, the subject's objective was to keep its current neural state as close as possible to the target state. To quantify how similar neural activity was to the target state, the neural distance metric 312 was used, which was the Euclidean distance between a subject's current neural state 310 and the target state 306 in subspace 304. A small neural distance 312 indicated the subject's current neural activity 310 was close to the target state 306. Also defined was a reward threshold 308 so that all neural distance values below the threshold were considered acceptable for the BCI task 320.

After completing the calibration task 310, the subject performed the BCI task 320, which contained alternating blocks of BCI and sham trials. In BCI trials, at time bin t, the computed neural distance 312 directly determined the shown annulus size in the following time bin. Therefore, the annulus size provided truthful moment-to-moment feedback on the subject's current neural state. At time bin t, if the subject's neural distance was small, the annulus would also be small in the following bin. Furthermore, in BCI trials, subjects were able to use neurofeedback to stabilize their neural activity over time. To incentivize them to do so, if the subject's neural activity was within the reward threshold 308 for 400 ms or 8 consecutive time bins, the trial ended and a liquid reward was given. Subjects had a 3.4 s neurofeedback period 402 to achieve this success criterion.

Sham trials were included as a control condition to determine the effects of genuine and accurate neurofeedback. For all sessions, there were two types of sham control trials: 1) Block Sham trials, which were organized in a homogeneous sham block, and 2) Interleaved Sham trials, which were randomly distributed within a BCI block, as shown in FIG. 3. In all sham trials, an approach was adopted wherein the visual feedback was disassociated from the subject's neural activity by replaying visual feedback displayed during BCI trials sampled from previous sessions. The BCI trials were specifically selected to show realistic but inaccurate visual feedback. To keep the subjects engaged in the task during sham trials, we provided a liquid reward as long as the animals maintained fixation for the length of the replay feedback period. See 404 in FIG. 4.

To assess the efficacy of neurofeedback from the BCI, the observed neural activity (which determines the task performance given the BCI mapping) was compared using accurate neurofeedback and sham feedback. Because block sham trials presented consecutive trials that always lasted the full 3.4 s of the feedback period, this set of trials was designated as a control group to determine the level at which subjects could suppress neural variability when not encouraged to control their neural activity. To evaluate how sham trials would have fared in our BCI task, the recorded neural activity during the sham trials of a session was played through its BCI decoder. For every sham trial, the trial was labelled as correct or incorrect by checking if there was any time bin at which the neural activity reached the BCI task success criterion. If the sham trial was deemed correct, only the bins from the start to when the success criterion was met for the following were considered for neural analyses, as shown in FIG. 5. After passing the recorded neural activity of sham trials through their respective session's BCI decoder, an assessment could be made as to how well the subject would have performed if those trials were treated as BCI trials. With this offline preprocessing step, BCI and sham trials were compared to determine whether subjects could use neurofeedback to volitionally control their PFC activity over time.

In a session, it was observed that subjects were better able to stabilize their neural activity in BCI blocks than they could in sham blocks (see line 606 in FIG. 6). To quantify how well the subjects performed in a trial, the bin-averaged neural distance, a continuous measure highly predictive of BCI task performance, was relied upon. On average, BCI blocks exhibited lower average neural distances than the sham blocks that immediately followed them (see horizontal lines in FIG. 6). Hence, subjects kept their neural activity closer to the target state in BCI blocks than in sham blocks. Across sessions, a similar trend was observed as BCI block trials had improved BCI task performance and lower neural distances than block sham trials (See FIGS. 7, 8 across all sessions). This neural and behavioral difference between the two conditions indicates that both subject monkeys could successfully use the BCI to maintain their neural activity near the target state.

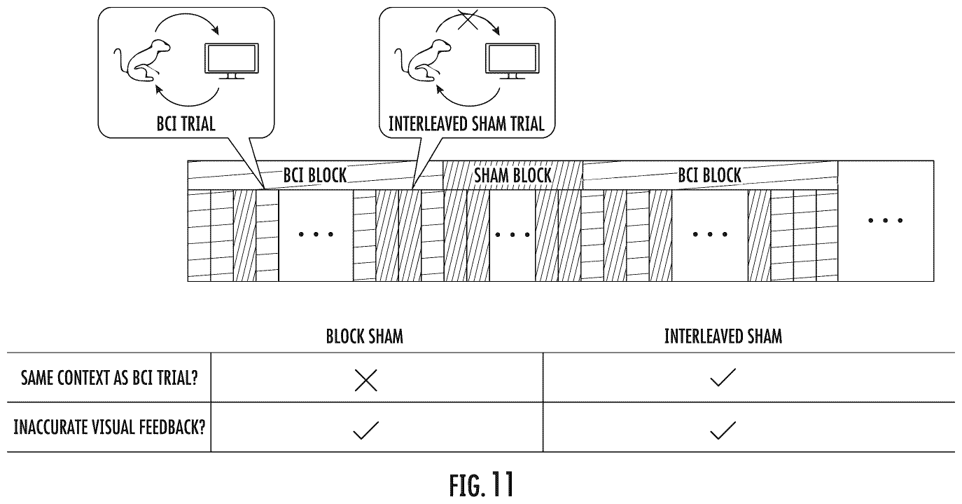

Subjects used moment-to-moment visual feedback from the annulus to reduce their neural distances. As shown in FIG. 11, there were two types of sham trials: block sham trials (sham trials in bolded red boxes) and interleaved sham trials (sham trials in bolded orange boxes). Both types of sham trials provide inaccurate feedback. However, in block sham trials, animals may have realized across trials that they were not in a BCI block and stopped trying. In interleaved sham trials, which are randomly placed in BCI blocks, subjects would expect to be in a BCI block and continue trying to bring their neural activity close to the target state. Thus, by comparing interleaved sham trials with BCI trials (BCI trials in bolded black boxes), it can be determined whether the subject relied on accurate real-time visual feedback to do better in BCI trials.

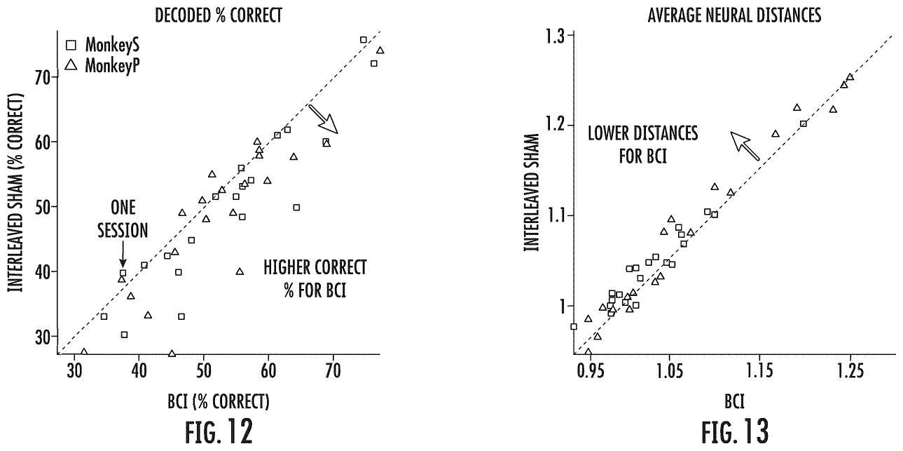

FIG. 12 shows a comparison of % correct trials for BCI and Interleaved sham trials. BCI % correct was higher than the correct percentage for Interleaved sham. FIG. 13 is a comparison of average neural distances for BCI and Interleaved sham trials. BCI sham had higher average neural distances than interleaved sham trials. Average neural distances for BCI trials were found to be above average neural distances for Interleaved sham trials for both subject monkeys.

Although BCI and block sham trials indeed differed in the validity of the visual feedback, they also differed in the context in which they appeared. During sham blocks, subjects encountered a set of consecutive trials that always lasted the entire length of the feedback period. The results could potentially be attributable not to an improvement in neural control with the BCI but rather to a gradual shift in PFC activity away from the calibration period. Such a shift could happen because of an increasing realization, and perhaps frustration, that the sham block consisted of numerous consecutive trials in which no control strategy could be successful. Thus, comparing the BCI trials with the block sham trials cannot alone demonstrate that the real-time visual neurofeedback provided by the annulus was successfully used by the subjects. To isolate the effects of presenting accurate moment-to-moment visual neurofeedback, BCI trials were compared with interleaved sham trials that were, unbeknownst to the subjects, randomly distributed in BCI blocks (See FIG. 11). Unlike block sham trials, interleaved sham trials occurred in the same context as BCI trials because, in both of these trial types, the subjects anticipated being in a trial with valid neurofeedback. The context did matter for the subjects as interleaved sham trials had higher decoded correct percentages and lower average neural distances than block sham trials. This result suggests that even when the shown feedback was incorrect, subjects kept trying to reduce their neural distances in interleaved sham trials, possibly by using an internal model to predict how their neural commands might drive the movements of the annulus. If the subjects did rely on the moment-to-moment visual neurofeedback to control their neural activity, a decreased task performance for the interleaved sham trials cold be predicted compared to their performance in BCI trials. That result was indeed discovered—in most sessions, subjects performed better during BCI trials than during interleaved sham trials (See FIG. 12). Furthermore, on average, subjects produced neural activity patterns with smaller neural distances during BCI trials than during interleaved sham trials (See FIG. 13). Taken together, these findings indicate that subjects directly used the real-time accurate neurofeedback from the annulus to help keep their neural activity close to the target neural state.

Subjects self-regulated an arousal-related internal state to help achieve BCI control. As noted earlier, internal states can fluctuate on both fast and slow timescales, which affects how neural population activity evolves. One such example is seen in the PFC, where neural fluctuations covaried with a slowly changing internal state linked to arousal. Under the BCI task paradigm, subjects must subdue these internal changes to help stabilize their neural activity.

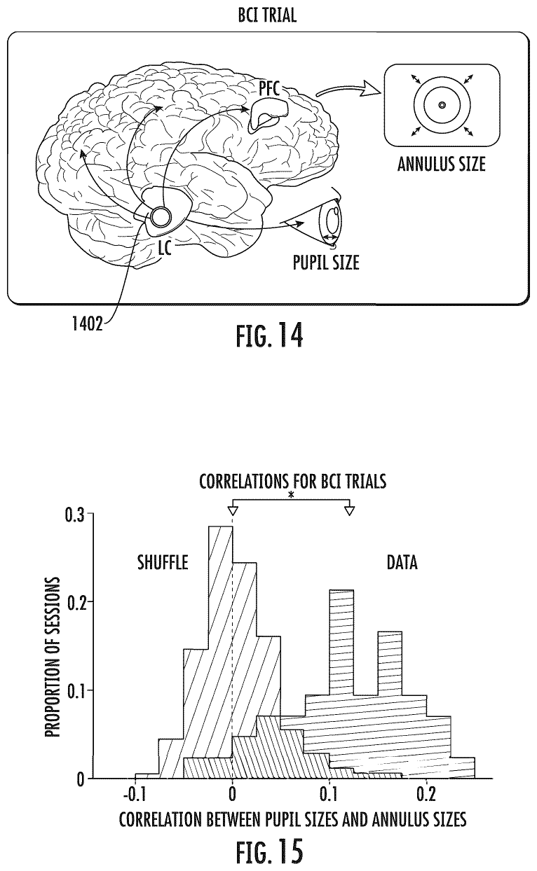

FIG. 14 shows that one mechanism by which the subject may suppress neural variability over time is through the control of an internal state. An upstream area of the PFC that is involved in regulating arousal is the Locus Coeruleus 1402. Could it be that the subjects are regulating their arousal levels to control this BCI? The LC 1402 also projects to the Edinger-Westphal nucleus (EW) which modulates arousal-related pupil size changes. To test this hypothesis, determine if there is any session-wide correlation over time between pupil sizes and the shown annulus sizes in BCI trials. FIG. 15 shows significantly positive correlations between pupil sizes and shown annulus sizes during BCI trials (dark grey histogram) compared to a chance distribution (light grey histogram) supporting this hypothesis. Each point in the observed histogram (dark grey) represents a Pearson correlation coefficient computed between the pupil size and shown annulus size for all bins belonging to BCI trials in a particular session. Upside-down triangles refer to the medians of each distribution.

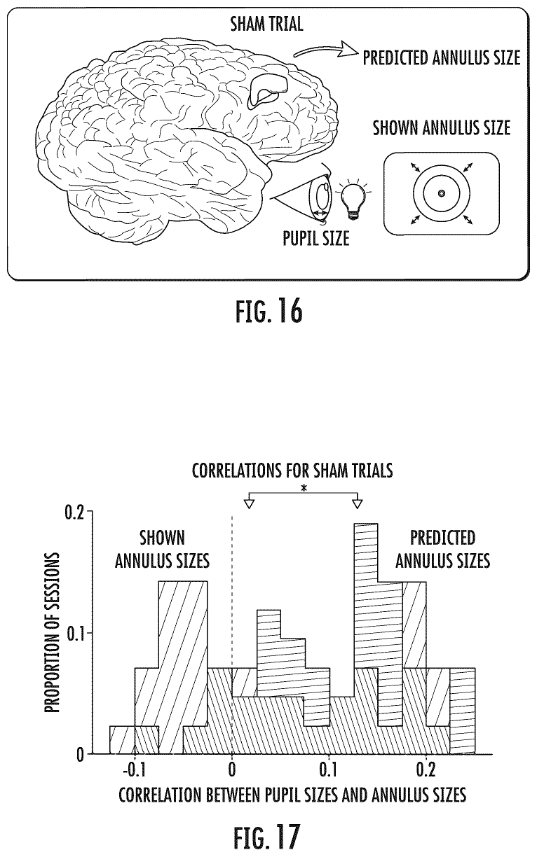

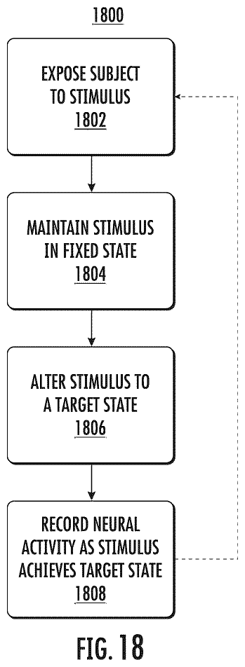

FIG. 16 shows that the correlation observed in BCI trials can also be explained by luminance-driven changes brought on by the size-varying annulus. These annulus changes may activate the pupillary light reflex which operates through the Olivary Pretectal Nucleus which subsequently projects to the EW to affect pupil size. To rule out this hypothesis, sham trials that have annulus sizes that can be predicted from recorded PFC activity (found using our decoder offline) and shown annulus sizes that correspond to the replay feedback can be analyzed. In BCI trials, the shown annulus size and the predicted annulus sizes will be exactly the same. FIG. 17 shows that predicted annulus sizes have more positive session-wide correlations with pupil sizes (dark grey histogram) than shown annulus sizes (light histogram) during sham trials which displayed replay feedback. This indicates that changes seen in pupil sizes during BCI trials can't only be explained by luminance and instead suggests the changes in pupil sizes are related to changes in arousal levels.

The relationship between the recorded PFC activity and changes in pupil size, a well-studied physiological measure of many different internal states, such as arousal were explored. Prior work has shown how the activity patterns of the locus coeruleus 1402 in FIG. 14, a brain area that mediates arousal through the global release of the neuromodulator norepinephrine covary over long and short timescales with pupil sizes. Furthermore, studies have shown how the dIPFC strongly innervates the LC and exhibits significant functional coupling with the LC in tasks involving cognitive control. Thus, it is reasonable to ask if moment-to-moment variations in the pupil size and shown annulus sizes were correlated within a session for BCI trials. Here, only the BCI trials were analyzed, as in those trials, the subjects received real-time neurofeedback about any changes they made in their pupil-linked internal states. It was found that during BCI trials, the shown annulus sizes were positively correlated with the subject's pupil size changes, suggesting that subjects mediated their arousal levels to keep their neural distances low. These correlations were significantly more positive than those expected from a shuffle control.

It is possible, however, that this positive correlation observed in BCI trials is induced primarily by the size-varying annulus' luminance changes, which can directly affect pupil sizes via the pupillary light reflex. A small annulus would concentrate a large amount of luminance at the fovea, causing the contraction of the pupil. To test whether this hypothesis, in which luminance solely drove pupil size changes, could explain our results, the correlations of the shown and predicted annulus sizes were compared with pupil size changes in sham trials (FIG. 16). BCI trials were not equipped to answer this question as, by definition, the shown and predicted annulus sizes were identical. Under this hypothesis, the correlations seen with the shown annulus sizes would be similar to those observed with the predicted annulus sizes. However, this is not the case—on average, the correlations involving the predicted annulus sizes were greater than those involving the shown annulus sizes (FIG. 17), indicating that luminance alone could not explain the positive correlations observed in the results involving BCI trials. In summary, these results indicate that one mechanism by which the subjects controlled the BCI was through the self-regulation of their arousal state.

As would be realized by those of skill in the art, specific exemplary embodiments disclosed herein, including specific equipment used, specific algorithms and specific visual stimuli are provided as exemplary embodiments and the invention is not meant to be limited thereby. Modifications and variations of the examples used herein are intended to be within the scope of the invention, which is given by the following claims:

1. A method comprising:
exposing a subject to a stimulus;
altering the stimulus toward a target state; and
recording neural activity of the subject as the stimulus achieves the target state to establish a baseline neural activity.

2. The method of claim 1 further comprising:
monitoring the neural activity of the subject as the subject is exposed to the stimulus during a time series of steps; and
for each time step, altering the stimulus to be closer to or farther away from the target state, based on a comparison of the neural activity observed in an immediately preceding time step to the baseline neural activity;
wherein the stimulus is altered to be closer to the target state if the neural activity observed in an immediately preceding time step moves closer to the baseline neural activity; and
wherein the stimulus is altered to be farther from the target state if the neural activity observed in an immediately preceding time step moves away from the baseline neural activity.

3. The method of claim 2 further comprising:
rewarding the subject when the observed neural activity is within a target threshold of the baseline for a predetermined number of time steps.

4. The method of claim 2 wherein the neural activity is monitored via a microelectrode array implanted in the brain of the subject.

5. The method of claim 4 wherein the microelectrode array is implanted in the prefrontal cortex anterior to the arcuate sulcus and dorsal to the medial sulcus in both hemispheres of the brain.

6. The method of claim 1 wherein the stimulus is a visual stimulus.

7. The method of claim 6 wherein the visual stimulus comprises altering a diameter of an annulus centered on a target dot.

8. The method of claim 2 wherein the comparison of the observed neural activity to the baseline neural activity is expressed as a neural distance metric.

9. The method of claim 2 wherein the time series of steps includes one or more time steps in which the visual feedback is unrelated to the observed neural activity.

10. The method of claim 2 further comprising:
holding the stimulus in a fixed state for a predetermined period of time prior to initiation of the time series of steps during which the observed neural activity controls the state of the stimulus.

11. A system comprising:
an electrode array implanted in the brain of a subject;
a neural network sorter trained to classify waveforms of neural activity collected by the microelectrode array as being caused by neural spiking or as being noise;
a processor; and
software that, when executed by the processor, performs the steps of:
exposing the subject to a stimulus;
monitoring neural activity of the subject as the subject is exposed to the stimulus during a time series of steps;
for each time step, calculating a neural distance metric for the neural activity observed in an immediately preceding time step;
altering the stimulus in the current time step based on the calculated neural distance.

12. The system of claim 11 wherein the neural distance metric quantifies a comparison of the observed neural activity to a baseline neural activity.

13. The system of claim 12 wherein altering the stimulus comprises:
altering the stimulus to be closer to a target state if the neural distance is smaller than the neural distance in the immediately preceding time step; and
altering the stimulus to be farther from the target state if the neural distance larger than the neural distance in the immediately preceding time step.

14. The system of claim 13 wherein the software performs the further steps of:
determining that the neural distance is within a within a target threshold of the baseline for a predetermined number of time steps; and
rewarding the subject.

15. The system of claim 11 wherein the software performs the further step of:
holding the stimulus in a fixed state for a predetermined period of time prior to initiation of the time series of steps.

16. The system of claim 12 wherein the target state is defined as a mean of neural activity observed during a calibration task, the calibration task comprising:
exposing a subject to a stimulus;
altering the stimulus; and
recording neural activity of the subject as the stimulus is altered to establish a baseline neural activity.

17. The system of claim 11 wherein the software performs the further steps of:
receiving signals from the microelectrode array;
performing bandpass filtering of the signals; and
digitizing the filtered signals.

18. The system of claim 17 wherein neural spiking is defined as a window of the filtered signal triggered by the signal crossing a predefined voltage threshold.

19. The system of claim 11 wherein the stimulus is a visual stimulus.

20. The system of claim 19 further comprising:
a display;
wherein the visual stimulus comprises altering a diameter of an annulus centered on a target dot, the target and the annulus being displayed on the display.
